(19) United States
(12) Patent Application Publication
(10) Pub. No.: US 2006/0052282 A1
(43) Pub. Date: Mar. 9, 2006

(54) Diagnostic and therapeutic means for pathologies associated with alpha 2 subunit of the na, k pump

(76) Inventors: Giorgio Casari, Milano (IT); Maurizio De Fusco, Milano (IT); Roberto Marconi, Milano (IT)

Correspondence Address: Albert Wai-Kit Chan;Law Offices of Albert Wai-Kit Chan, World Plaza Suite 604, 141-07 20th Avenue, Whitestone, NY, 11357, US

(21) Appl. No.: 10/535,437

(22) PCT Filed: Nov. 12, 2003
(86) PCT No.: PCT/EP03/12635

§ 371 Date: Aug. 15, 2005

(30) Foreign Application Priority Data
Nov. 15, 2002 (IT) RM2002A000576

(51) Int. Cl. A61K 38/17; C12Q 1/68; C07H 21/04; C12P 21/06; C07K 14/705

(52) U.S. Cl. 514/2; 435/6; 536/23.5; 530/350; 435/69.1; 435/320.1; 435/325

(57) ABSTRACT

A nucleic acid is described that comprises at least one segment of the gene encoding a functional segment of the alpha 2 subunit of the Na,K pump (ATPase, ATP1A2) for use in the diagnosis or treatment of pathologies associated with migraine or with alternating hemiplegia of the childhood. Appropriate diagnostic kits and methods to identify agonist or antagonist agents are also described.

The present invention concerns diagnostic and therapeutic means for pathologies associated with alpha 2 subunit of the Na,K pump, as migraine and alternating hemiplegia of the childhood.

Migraine is characterized by headache attacks and is sometimes associated with autonomic nervous system dysfunctions and transient neurological symptoms (aura). The prevalence of migraine in the general population is 12%, and 20% of patients present with aura (1-5).

Although the mode of transmission is controversial (6), migraine shows strong familial aggregation (7). Several population-based and twin-based studies have indicated that genetic factors are implicated, particularly in migraine with aura (8, 9). Familial hemiplegic migraine (FHM) is a disabling neurological disease that manifests with aura and hemiparesis. It affects about 1/10,000 to 1/50,000 individuals and is transmitted as an autosomal dominant trait (10).

A gene associated with FHM1(MIM 141500) and encoding a neuronal calcium channel protein (CACNA1A) has previously been identified (11). The PCT patent application WO98/55647 describes an indirect genotyping method for diagnosing hemiplegic migraine type 2 that concerns a wide region of 21 cM (centimorgan) of chromosome 1q21-23. The description does not identify any genes associated with the disease but suggests two candidate genes, GIRK3 (encoding a potassium channel protein) and CACNL1A6 (encoding a calcium channel protein).

The authors of the present invention have identified the gene associated with FHM2 (MIM 602481) that maps on chromosome 1q23 (12) and have shown that mutations in the alpha 2 subunit of the Na,K ATPase pump (ATP1A2) are responsible for the disease. The identified gene does not correspond to any genes or regions suggested in the previous technical documentation, particularly as regards the aforesaid patent application WO98/55647. The authors have demonstrated that the identified missense mutations cause a loss-of-function of the major ion transport system. This has relevant implications for the origin of cortical spreading depression of neuronal disease and the development of migraine. It is the first demonstration that mutations in the Na,K pump are associated with genetic diseases. It should also be stressed that a study on GIRK3 and CACNL1A6 have not shown any mutations in these genes that could be correlated with migraine.

Furthermore, it is possible to study polymorphisms associated with said gene to correlate them as predisposing factors for common migraine.

Alternating hemiplegia of the childhood (AHC, OMIM 104290) is a rare syndrome (estimated prevalence 1 in 1.000.000), characterized by early onset of episodic hemi- or quadriplegia lasting minutes to days.

Mutation analysis in the ATP1A2 gene was performed by direct sequencing of all exons with the same primers used for amplification. An heterozygous mutation (1237 C−>A) segregating with the disease in a AHC family and causing a threonine to asparagine replacement (T378N) was found. This mutation is not present in any of the unaffected members of the family and in 250 control chromosomes.

Hence, the object of the present invention is a nucleic acid comprising at least one segment of the gene encoding a functional portion or gene-regulating region of the alpha 2 subunit of the Na,K pump (ATPase, ATP1A2) for use in the diagnosis of pathologies associated with migraine or with the alternating hemiplegia of the childhood.

A further object of the invention is a nucleic acid comprising at lease one segment of the gene encoding a functional portion or gene-regulating region of the alpha 2 subunit of the Na,K pump (ATPase, ATP1A2) for use in genetic therapy for pathologies associated with migraine or with the alternating hemiplegia of the childhood.

A further object of the invention is a method to detect in an individual at least one mutation of the gene encoding the alpha 2 subunit of the Na,K human pump (ATPase, ATP1A2) located on chromosome1, associated with migraine disorders or with the alternating hemiplegia of the childhood, which comprises the steps of:—collecting a sample containing a sufficient quantity of the individual's DNA or that is reproducible in culture;—isolating the DNA from the sample;—exponentially amplifying the DNA using as an oligonucleotide pair for the amplification reaction at least two oligonucleotides that are able to amplify at least one segment of the gene encoding the alpha 2 subunit of the Na,K human pump (ATPase, ATP1A2) or of a segment of the region regulating it;—detecting in at least one amplified segment any mutations compared with a healthy control. Preferably, the oligonucleotide pairs are:

17 AGTCCCTCTGACCTCCCTGAT CCACTGTGCCATCACGATT
19 CTTCTGCTTCCTGCTCTGACC ACACATGTGCGCTGTGTTTAC.

In an embodiment of the method of invention, the DNA exponential amplification phase is performed using oligonucleotide pairs that are able to amplify the entire encoding portion of the gene encoding the alpha 2 subunit of the Na,K human pump (ATPase, ATP1A2). Preferably, the DNA exponential amplification phase to amplify the entire encoding portion of the gene encoding the alpha 2 subunit of the Na,K human pump (ATPase, ATP1A2) comprises the use of at least one of the following oligonucleotide pairs:

| | | |
|---|---|---|
| 1 | TGTTGCTTTGGCTTTCTCTGT | CTCCCTCACCCTCTAGACTGC |
| 2 + 3 | CCCCTCTCTTCCCTGACTCT | GCCTCTTTTGTTCCTTCCCTA |
| 4 | ATGGTGACTGGCTGGGTTG | CAGGGTTGGAGGACAGTCAC |
| 5 | AGCTGCCCCTTTAGGGTTG | ACCTTACAGCCTAGCCCAGAG |
| 6 | GAGACCAGCAGGAGAAGAAGG | AGACTCAACTGCTTGCTCTGG |
| 7 | TACAAGTGGCTCTGCCAGTCT | AGCCCTTCATCCTGACTATGG |
| 8 | CAGGAAATAGGATGGGACTGC | GTAGTGAGACCCTCCCCTGGT |
| 9 | ATCTCCGGCTTCAGCCTTAAC | TAATCCTATCCACCCCCTCTG |
| 10 + 11 | CTCCTGGTTCCCCCTCAT | TCCCTCTCTCTTCCTCTGTCC |
| 12 | GCGCTACCAAGACAAGTATGG | CTTGGGAATCCCCTTCTGAG |
| 13 | GAAGCCACTCTGCGGATCT | ACTGCAGCTCCTTGAACTCTG |
| 14 | GGAGGGGGATAAACCCTTAAT | GACGTGTTGATTAGGGCACAG |
| 15 | AGGGGTCAGCTGTCTCTGTC | GGTCCCTGCCTGTCATCTG |
| 16 | AAGGGGTTTCGTCCTCAAGT | TCAGTATCCTGCAAACCATCC |
| 17 | AGTCCCTCTGACCTCCCTGAT | CCACTGTGCCATCACGATT |
| 18 | TCATCTCCTACGTCCCTTCAA | AGCTGGGAAAAGAACCCTGT |
| 19 | CTTCTGCTTCCTGCTCTGACC | ACACATGTGCGCTGTGTTTAC |
| 20 | CCTCCGACACTCTCATCTGTC | CTGTGTGGGTTGGTGAGTGT |
| 21 | CTTCACCTGCCACCTCCTT | CCCCCGTATGACTACTCAGG |
| 22 | CGCTTTGAATGCTCCTTTATG | GAGGGAGGAGCTGGTGGT |
| 23 | GCCTCCTTTTAAGCTCATGCT | GCCTCATTATCTCTCCCCAAA |

In an embodiment of the method of invention, the DNA exponential amplification phase is performed using oligonucleotide pairs that are able to amplify the regulating region of the gene encoding the alpha 2 subunit of the Na,K human pump (ATPase, ATP1A2). Preferably, the DNA exponential amplification phase to amplify the regulating region of the gene encoding the alpha 2 subunit of the Na,K human pump (ATPase, ATP1A2) comprises the use of the following oligonucleotide pairs:

1_Pr TTCCCCTCACTCCATCTCTG GACCCCTGCTCTTTAGGGATA
2_Pr GA1TCAGGACCACTCCATCC GGGAACAGTCAGAGGACAGG

In a preferred embodiment of the method of the invention, the detection phase of at least one amplified segment with any mutations compared with a healthy control is performed using direct sequencing or an SSCP method (single strand conformation polymorphism) (17) DHPLC or DGGE (denaturing gradient gel electrophoresis) (18) or other methods known to an expert from the field.

A further object of the invention is a diagnostic kit for pathologies associated with migraine or with alternating hemiplegia of the childhood which comprises:—at least a pair of oligonucleotides for the exponential amplification reaction of at least one segment of the gene encoding the alpha 2 subunit of the Na,K human pump (ATPase, ATP1A2), in which the segment encodes a functional portion or a gene-regulating portion of the subunit;—a control DNA from a non affected individual. In a preferred form, the oligonucleotide pairs for the amplification reaction are able to amplify the entire encoding region of the gene encoding the alpha 2 subunit of the Na,K human pump (ATPase, ATP1A2).

A further object of the invention is the alpha 2 subunit protein of the Na,K human pump (ATPase, ATP1A2) or a functional portion thereof for use in the diagnosis of pathologies associated with migraine or with alternating hemiplegia of the childhood.

A further object of the invention is the alpha 2 subunit protein of the Na,K human pump (ATPase, ATP1A2) or a functional portion thereof for use in the treatment of pathologies associated with migraine or with alternating hemiplegia of the childhood.

A further object of the invention is a method for the identification of an agonist or antagonist agent of the Na,K human pump (ATPase, ATP1A2) or a functional portion or a gene-regulating portion of thereof, that comprises:
(i) transfection of a cell line with a gene for a mutant isoform of the Na,K human pump (ATPase, ATP1A2) resistant to oubain;
(ii) appropriate exposure of the transfected cells to the agent;
(iii) measurement of the Na,K pump activity in relation to ion transport with labelled ions.

A further object of the invention is a method to identify an agonist or antagonist agent of the Na, K pump (ATPase, ATP1A2) or a functional portion, that comprises the phases:
(i) use of an agent to treat a transgenic animal that expresses a mutant isoform of the Na,K pump (ATPase, ATP1A2) or that partially or completely deletes the gene encoding the Na,K pump (ATPase, ATP1A2) or
(ii) use of an agent to treat eukaryotic or prokaryotic cell lines that express mutant or normal forms of the Na,K pump (ATPase, ATP1A2) by transient or stable transfection or in physiological conditions.

The invention is described below in reference to non limiting examples and the following figures:

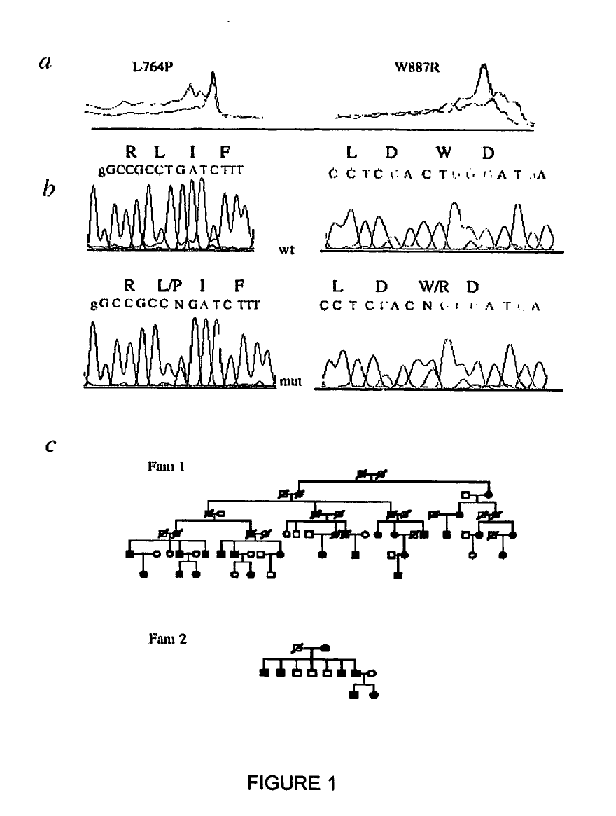

FIG. 1. ATP1A2 mutation detection. Panel a, the normal (blue) and mutant (red) D-HPLC elution patterns of exon 17 (left) and 19 (right); Panel b shows the direct-sequencing electropherograms of the control (upper part) and mutant heterozygotes (lower part); Panel c, the pedigree of the two FHM2 families.

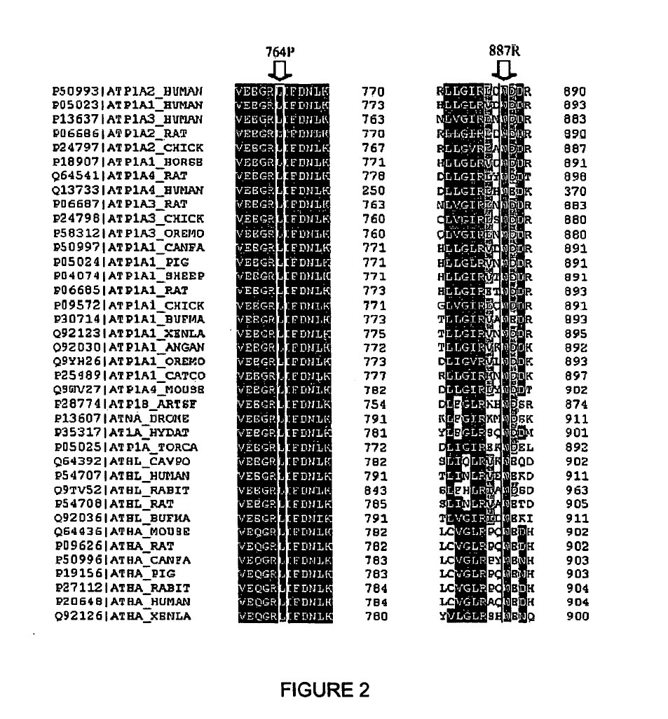

FIG. 2. Local amino acid sequence alignment of ATPases. The complete conservation of L764 (left) and W887 (right) in several subunits of Na,K ATPases and H,K ATPases is shown. The relative SwissProt accession number is indicated.

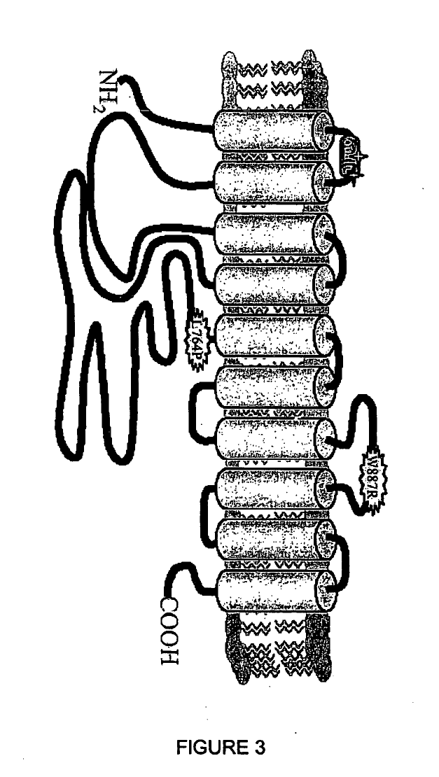

FIG. 3. ATP1A2 protein topology. The ouabain binding site on the first loop (M1-M2; asterisks indicate the mutagenized amino acids to confer ouabain resistance) and the two mutations on the largest intracellular (M4-M5) and extracellular (M7-M8) loops are highlighted.

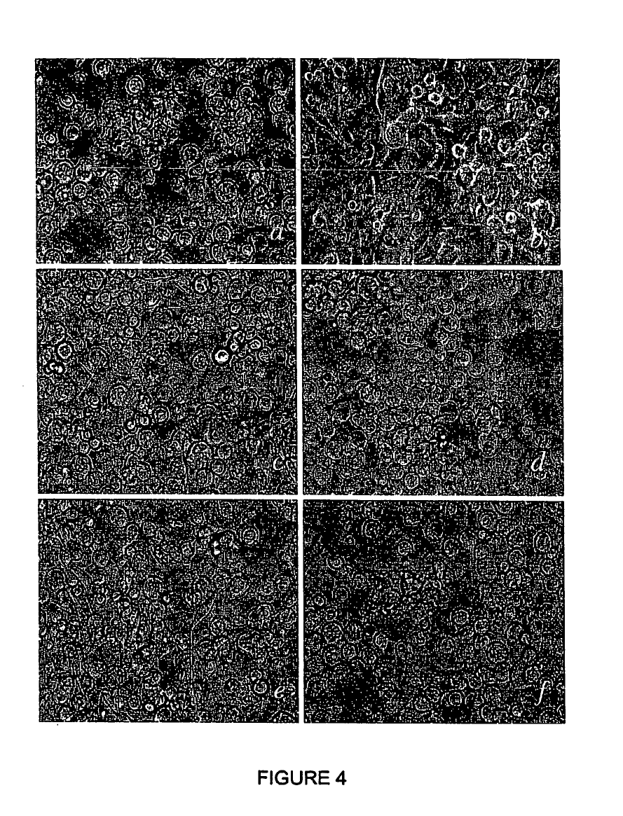

FIG. 4. Ouabain treatment of transfected HeLa cells. Phase contrast pictures of HeLa cells taken after 36 hr of 1 μM ouabain challenge transfected with: panel a, mock transfected cells (a construct expressing wild-type ATP1A2 non-ouabain resistant was used); panel b, a ouabain resistant wild-type ATP1A2, pA2Oua^{r}-wt; panels d and f, ouabain resistant ATP1A2 mutants, pA2Oua^{r}-P764 and pA2Oua^{r}-R887, respectively; panel c, a 1:1 mixture of pA2Oua^{r}-wt+pA2Oua^{r}-P764, to simulate the L764P heterozygous state; panel e, a 1:1 mixture of pA2Oua^{r}-wt+pA2Oua^{r}-R887, to simulate the W887R heterozygous state. All experiments were performed by co-transfecting an ATP1B2 expressing construct.

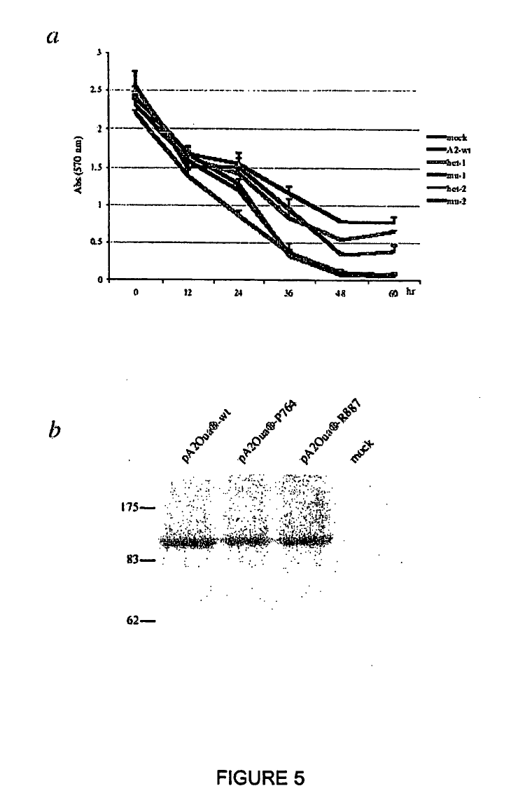

FIG. 5. Time course of ouabain toxicity. Panel a, cell viability by MTT assay of HeLa cells transfected with different constructs as reported in FIG. 4: mock; A2-wt (pA2Oua^{r}-wt); mu-1 (pA2Oua^{r}-P764); het-1 (pA2Oua^{r}-wt+pA2Oua^{r}-P764); mu-2 (pA2Oua^{r}-R887); het-2 (pA2Oua^{r}-wt+pA2Oua^{r}-R887). Both mutants and simulated heterozygotes are significantly different from A2-wt (at least P<0.04). Bars represent SD. Panel b, in vitro transcription and translation confirming the expected molecular mass of ATP1A2 protein of 112 kDa.

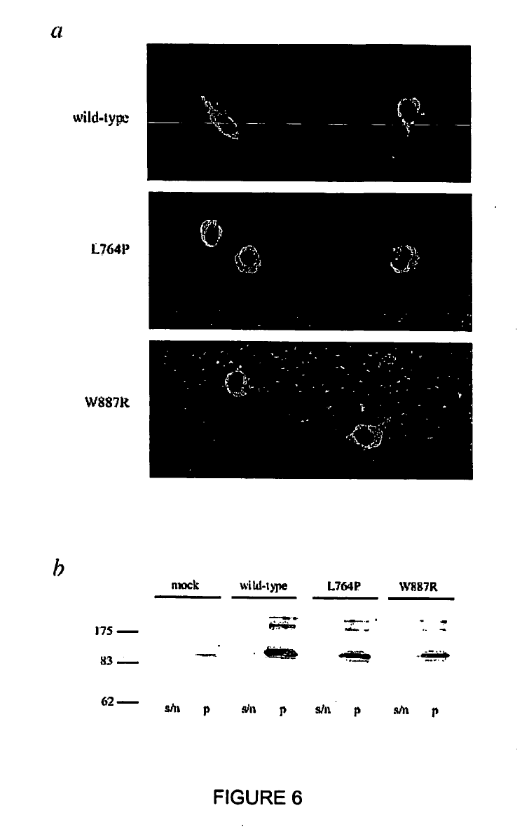

FIG. 6. Localization of mutant ATP1A2 to the plasma membrane. Panel a, immunocytochemistry on COS-7 cells of the c-myc-derivatives, pA2Oua^{r}-wt-myc, pA2Oua^{r}-P764-myc and pA2Oua^{r}-R887-myc, showing the plasma membrane localization of both wild type and mutant isoforms. Panel b, subcellular fractionation of transfected COS-7 cells demonstrating the plasma membrane co-sedimentation with ATP1A2 c-myc-derivatives; s/n, supernatant; p, pellet.

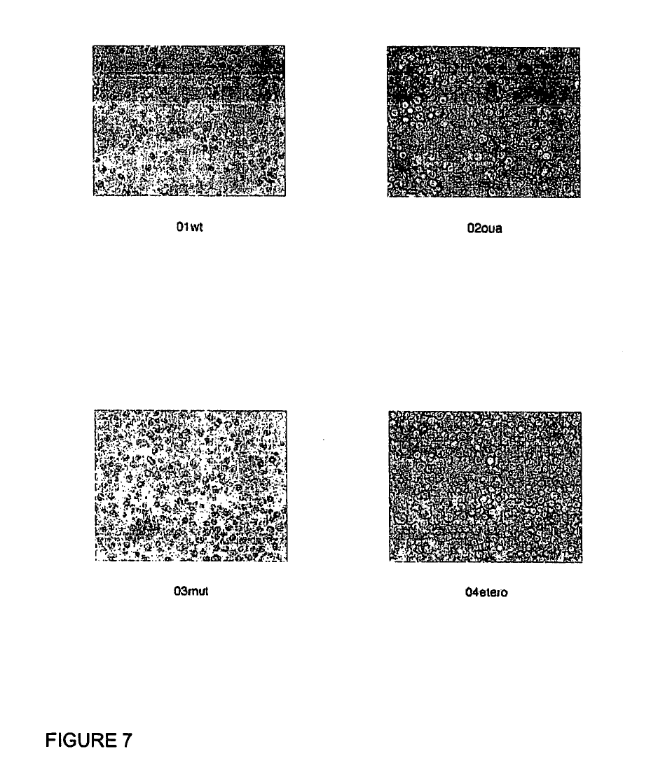

FIG. 7. Phase-contrast pictures of transfected HeLa cells taken after 72 h of treatment with 1 μM ouabain. a, transfection with a cDNA construct expressing non-ouabain-resistant wild-type ATP1A2. b, transfection with a cDNA construct expressing ouabain-resistant wild-type ATP1A2. c, transfection with a cDNA construct expressing ouabain-resistant T328N ATP1A2 mutant. d, transfection with a 1:1 mix of constructs expressing ouabain-resistant wild-type ATP1A2 and T328N ATP1A2 mutant to simulate the hetrozygous state.

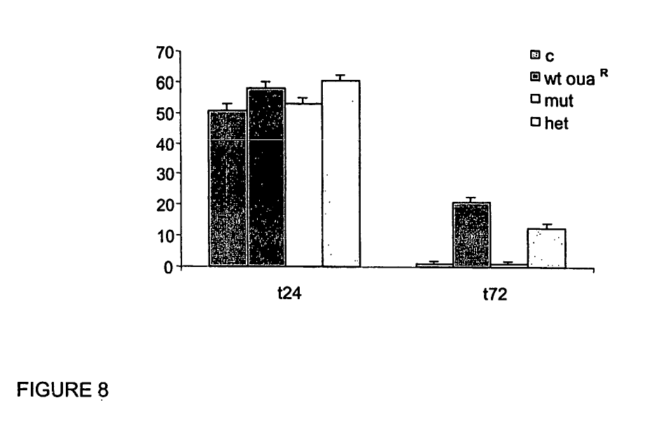

FIG. 8. Cell viability assessed by MTT assay of the transfected HeLa cells shown in FIG. 7. Y axis represents the percentage of surviving cells

### EXAMPLE 1

### Migraine

Materials and Methods

FHM2 Families

Twenty-two subjects from a large Italian pedigree (family 1), originating from Tuscany, with a clinical diagnosis of FHM (5) and seven members with similar manifestations from an unrelated pedigree (family 2) from Sicily, were selected. No cerebellar signs were associated with FHM. The onset of attacks always occurred by the age of twenty. Additional features were history of seizures in five members (three subjects from family 1 and two subjects from family 2) and mild or moderate mental retardation in two subjects from family 1. Two hundred randomly collected healthy individuals from the Italian population were used as control subjects.

Mutation Screening

We determined the genomic organization of the human ATP1A2 gene by aligning the sequence of ATP1A2 mRNA (AC NM_{—}000702) with the corresponding genomic sequence on clone RP11-536C5. Designed oligonucleotide primers for amplification of the gene encoding regions are reported in Table 1. PCR products that showed an abnormal D-HPLC (Wave, Transgenomic, Crewe, UK) retention patterns were subjected to direct sequencing (DYEnamic ET Dye Terminator Kit, Amersham Biosciences, Piscataway, N.J., USA).

TABLE 1

| | | | |
|---|---|---|---|
| Exon | forward primer | reverse primer | bp |

| | | | |
|---|---|---|---|
| 1 | TGTTGCUTGGCTTTCTCTGT | CTCCCTCACCCTCTAGACTGC | 177 |
| 2 + 3 | CCCCTCTCTTCCCTGACTCT | GCCTCTTTTGTTCCTTCCCTA | 423 |
| 4 | ATGGTGACTGGCTGGGTTG | CAGGGTTGGAGGACAGTCAC | 316 |
| 5 | AGCTGCCCCTTTAGGGTTG | ACCTTACAGCCTAGCCCAGAG | 213 |
| 6 | GAGACCAGCAGGAGAAGAAGG | AGACTCAACTGCTTGCTCTGG | 238 |
| 7 | TACAAGTGGCTCTGCCAGTCT | AGCCCTTCATCCTGACTATGG | 234 |
| 8 | CAGGAAATAGGATGGGACTGC | GTAGTGAGACCCTCCCCTGGT | 385 |
| 9 | ATCTCCGGCTTCAGCCTTAAC | TAATCCTATCCACCCCCTCTG | 283 |
| 10 + 11 | CTCCTGGTTCCCCCTCAT | TCCCTCTCTCTTCCTCTGTCC | 487 |
| 12 | GCGCTACCAAGACAAGTATGG | CTTGGGAATCCCCTTCTGAG | 284 |
| 13 | GAAGCCACTCTGCGGATCT | ACTGCAGCTCCTTGAACTCTG | 286 |
| 14 | GGAGGGGGATAAACCCTTAAT | GACGTGTTGATTAGGGCACAG | 236 |
| 15 | AGGGGTCAGCTGTCTCTGTC | GGTCCCTGCCTGTCATCTG | 284 |
| 16 | AAGGGGTTTTCGTCCTCAAGT | TCAGTATCCTGCAAACCATCC | 284 |
| 17* | AGTCCCTCTGACCTCCCTGAT | CCACTGTGCCATCACGATT | 252 |
| 18 | TCATCTCCTACGTCCCTTCAA | AGCTGGGAAAAGAACCCTGT | 234 |
| 19* | CTTCTGCTTCCTGCTCTGACC | ACACATGTGCGCTGTGTTTAC | 232 |
| 20 | CCTCCGACACTCTCATCTGTC | CTGTGTGGGTTGGTGAGTGT | 236 |
| 21 | CTTCACCTGCCACCTCCTT | CCCCCGTATGACTACTCAGG | 176 |
| 22 | CGCTTTGAATGCTCCTTTATG | GAGGGAGGAGCTGGTGGT | 223 |
| 23 | GCCTCCTTTTAAGCTCATGCT | GCCTCATTATCTCTCCCCAAA | 206 |

The primer pairs 17 and 19 (*) were used to identify the two mutations associated with FHM2. The PCR were designed with a uniform annealing temperature of 57° C.

The oligonucleotides that permit the amplification of the gene expression regulating regions (about 3 kb, subdivided in two partially overlapping segment) are listed in Table 2.

TABLE 2

1_Pr TTCCCCTCACTCCATCTCTG GACCCCTGCTCTTTAGGGATA
2_Pr GATTCAGGACCACTCCATCC GGGAACAGTCAGAGGACAGG

The analysis of the amplified DNA was performed using direct sequencing and DHPLC (denaturing high-pressure chromatography (16).

Constructs and Site-Directed Mutagenesis

The full-length cDNA coding for the beta 2 (ATP1B2, NM_{—}001678) and alpha 2 were derived from IMAGE clone 23453 and DFKZp761 D047, respectively, and subcloned in the expression vector pcDNA3.1 (Invitrogene, Carlsbad, Calif., USA). We used the QuickChange site-directed mutagenesis kit (Stratagene, La Jolla, Calif., USA) for mutagenizing the ATP1A2 cDNA as follows:
a) nt451 A>G (Q116R) and nt483 A>G (N127D) to obtain the construct pA2Oua^{r}-wt expressing the ouabain-resistant isoform;
b) nt2395 T>C (L764P) on pA2Oua^{r}-wt, obtaining pA2Oua^{r}-P764;
c) nt2763 T>C (W887R) on pA2Oua^{r}-wt, obtaining pA2Oua^{r}-R887.
d) c-myc-tagging the ATP1A2 cDNA. Expression constructs (pA2Oua^{r}-wt, pA2Oua^{r}-P764, and pA2Oua^{r}-R887) were mutagenized by replacing the original start codon with the c-myc tag (consisting of aa MAEEQKLISEEDL, corresponding to aa 408419 of the human c-myc AC 0907235A) obtaining pA2Oua^{r}-wt-myc, pA2Oua^{r}-P764-myc and pA2Oua^{r}-R887-myc. All constructs were sequence-verified.
   In Vitro Transcription and Translation

In vitro transcription and translation was performed using the TNT Coupled Reticulocyte Lysate System (Promega, Madison, Wis., USA) in the presence of 20 microCi [³⁵S] methionine (1000 Ci/mmole) and neosynthesized proteins were separated by SDS/PAGE (8%).

Electrophoresis and Western Blot Analysis

Equal amounts of proteins were resuspended in SDS-PAGE buffer (62.5 mM Tris-HCl pH 6.8, 2% SDS, 10% glycerol, 5% 2-mercaptoethanol) and separated for 2 h at 100V in 10% SDS-polyacrylamide gels. Transblotted nitrocellulose membranes were incubated with monoclonal primary antibodies anti c-myc 9E10 (10 μg/ml) or polyclonal antibodies anti-Integrin beta 1 followed by incubation with horseradish peroxidase (HRP)-conjugated secondary antibodies. Protein bands were visualized with the Enhanced Chemiluminescence kit (Amersham Biosciences, Piscataway, N.J., USA).

Transfections and Ouabain Treatment

Constructs were transfected by calcium phosphate (HeLa cells) following standard procedures or by Lipofectamine (COS-7 cells), (Gibco, Invitrogene Co., Carlsbad, Calif., USA).

Cell viability was measured with the MTT (13) reduction assay after 12, 24, 36, 48 and 60 hours. Each experiment was made in triplicate and statistical analysis was done by Student's t-test (homoscedastic).

Immunocytochemistry

After transfection (48 hr), COS-7 cells were fixed in 100% methanol and incubated with monoclonal primary antibodies anti c-myc 9E10. Cells were then washed with PBS and incubated with Alexa Fluor 488-conjugated anti-mouse secondary antibody (Molecular Probes, Eugene, Oreg., USA). Cells were coverslipped in fluorescent mounting medium (DAKO, Glostrup, Danmark) and visualized under epifluorescence optics.

Subcellular Fractionation

COS-7 cells were lysated on ice in 0.5 M NaCl, 10 mM NA2C03, 0.1 mM PMSF, 10 μg/ml Aprotenin, 10 μg/ml Leupeptin, homogenated and centrifuged at 2000 g for 20 min at 4° C. to discard nuclei and cellular debris. Separation of the membrane fraction (pellet) from the cytosolic fraction (supernatant) was achieved by centrifugation at 100,000 g for 40 min at 4° C. in a Beckman TL 100 ultracentrifuge.

Results

Mutation Analysis

Although the reduced FHM2 critical region spanned only 0.9 Mb between D1S2635 and CASQ1-SNP, several positional candidate genes, which are expressed in the central nervous system, are present in this genomic area. We performed mutation analysis on two probands of the FHM2 families by both D-HPLC (denaturing HPLC) and direct sequencing on the two potassium channel genes, KCNJ9 and KCNJ10 and the CASQ1 gene coding for calsequestrin with negative results.

In contrast, D-HPLC mutation scanning of the ATP1A2 gene encoding the alpha 2 subunit of the Na,K ATPase gave aberrant elution patterns for exon 17 and exon 19 in families 1 and 2, respectively (FIG. 1a). Sequencing analysis revealed the presence of two point mutations (nt 2395 T to C and nt 2763 T to C; FIG. 1b), each segregating with the disease in the respective families (FIG. 1c) and causing the amino acid replacements leucine to proline (L764P) in family 1 and tryptophan to arginine (W887R) in family 2. Both missense mutations were absent in 400 control chromosomes. L764 and W887 are completely conserved among alpha subunits from several evolutionary distant species (FIG. 2), thus strongly suggesting a causal role of ATP1A2 mutations in the pathogenesis of FHM2.

The Na,K pump is a heterodimeric structure formed by a large catalytic alpha subunit and a small ancillary beta subunit. The alpha subunits traverse the plasma membrane with 10 transmembrane segments (M1-M10) (14) and expose the amino- and carboxy-termini towards the cytoplasm. This configuration assigns five extracellular and four intracellular loop domains. L764P and W887R mutations have different localization within the alpha topology: L764P maps to the large intracellular loop between M4 and M5, while W887R localizes to the apical M7-M8 loop (FIG. 3).

Functional Evidence of Impaired Ion Transport

In order to evaluate the functional consequences of these two amino acid replacements involving different protein structures, we carried out various transfection experiments. We cloned the full-length human cDNAs of the alpha 2 and beta 2 subunits (see Materials and Methods section). Since both subunits are required for assembling the active alpha-beta heterocomplex, all transfection experiments hereafter were carried out by co-transfecting alpha 2 and beta 2 constructs with equal stoichiometry. By introducing the mutant isoforms (i.e. expressing the mutant P764 and R887 full-length cDNAs), we obtained no significant changes in the cell shape or growth rate (data not shown), thus excluding a primary dominant-negative effect.

As all vertebrate cells present Na,K ATPase activity, the endogenous Na,K pump activity of HeLa cells was quenched to test the ion transport performance of the exogenous mutant forms. A site-directed mutagenesis was carried out to abolish the natural ouabain sensitivity of the ATP1A2 construct by mutagenizing two amino acids, Q116R and N127D, in the first extracellular loop that is part of the ouabain binding site of Na,K ATPase (15). HeLa cells transfected with the ouabain-resistant ATP1A2 cDNA construct (pA2Oua^{r}-wt) can survive and grow in 1 μM ouabain-containing media (FIG. 4, panel b), while mock transfected cells die within 36-48 hours (FIG. 4, panel a). Identical results were obtained with the human cell line HEK293.

Once positively tested for ouabain resistance, the pA2Oua^{r}-wt construct was subsequently mutagenized to introduce the FHM2 mutations L764P and W887R, obtaining the corresponding constructs pA2Oua^{r}-P764 and pA2Oua^{r}-R887. HeLa cells transfected with pA2Oua^{r}-P764 or pA2Oua^{r}-R887 failed to survive 1 μM ouabain treatment (FIG. 4, panels d and f), thus suggesting that both L764P and W887R are loss-of-function mutations.

Simulated heterozygous states obtained by co-transfecting equal amounts of wild-type and mutant constructs showed an intermediate behavior (FIG. 4, panel c and e). Both mutant ATP1A2 isoforms showed early cell mortality typical of cells lacking Na,K pump activity (FIG. 5a). To exclude the possible production of aberrant proteins from site-directed mutagenesis, we tested the wt and mutant constructs by in vitro transcription and translation experiments and direct sequencing. As shown in FIG. 5b, all three constructs gave the expected 112 kDa protein band, thus excluding the possibility that a cloning artifact is responsible for the vulnerability of HeLa cells to ouabain when transfected with the mutant ATP1A2 cDNAs.

Mutant ATP1A2 Isoforms are Delivered to the Plasma Membrane

We investigated the subcellular localization of the two mutant isoforms. The three constructs (pA2Oua^{r}-wt, pA2Oua^{r}-P764, and pA2Oua^{r}-R887) were engineered by adding a 5′ tag coding for the c-myc epitope and transfected into COS-7 cells. FIG. 6a shows the expected immunofluorescence localization to the plasma membrane of all isoforms, both wild type and the two mutants. Subcellular fractionation confirmed the physiological location in the membrane fraction (FIG. 6b), where the integrin beta 1 subunit was detected as a control.

These data demonstrate that both missense mutations are independently sufficient to inhibit Na,K pump activity, without affecting the assembling with the beta subunit and the complex translocation to the cell membrane.

### EXAMPLE 2

### Alternating Hemiplegia of the Childhood

Materials and Methods

Constructs and Site-Directed Mutagenesis.

The full length cDNA coding for the alpha 2 was subcloned in the expression vector pcDNA3.1 (Invitrogene, Carlsbad, Calif., USA). We used the QuickChange site-directed mutagenesis kit (Stratagene, La lolla, CA, USA) for mutagenizing the ATP1A2 cDNA as follows:
Q116R and N127D to obtain the construct pA2Oua^{r}-wt expressing the ouabain resistant isoform;
T378N on pA2Oua^{r}-wt, obtaining pA2Oua^{r}-AHC mutated form;
All constructs were sequence-verified.
   Transfections and Ouabain Treatment.

Constructs were transfected by calcium phosphate (HeLa cells) following standard procedures.

Cell viability was measured with the MTT reduction assay after 24,48 and 72 hours of 1 μM ouabain challenge. We performed two independent experiments and in each experiment, datapoints are in triplicate.

Results

Alternating hemiplegia of the childhood (AHC, OMIM 104290) is a rare syndrome (estimated prevalence 1 in 1.000.000), characterized by early onset of episodic hemi- or quadriplegia lasting minutes to days.

Mutation analysis in the ATP1A2 gene was performed by direct sequencing of all exons with the same primers used for amplification. An heterozygous mutation (1237 C−>A) segregating with the disease in a AHC family and causing a threonine to asparagine replacement (T378N) was found. This mutation is not present in any of the unaffected members of the family and in 250 control chromosomes.

This missense mutation localizes to the ATPases phosphorylation site (DKTGTLT, aa 374-380) of the hydrolase domain of the protein. In the □□ subunit topology the mutated residue resides in the large intracellular loop within the M4-M5 transmembrane segments (M1-M1 0, [Hu, 2000 #7823]). The affected residue is highly conserved in all the known □ subunits of the Na,K pump from vertebrates to invertebrates suggesting a functional role in pump activation.

To evaluate the functional consequences of this mutation we carried out transfection experiments in human HeLa cells. Since all mammalian cells have Na+/K+ ATPase, we quenced the endogenous pump activity using ouabain, and tested the function of the exogenously transfected mutant of cDNA constructs engineered to be resistant to ouabain. Using site-directed mutagenesis we introduced two amino acid changes (Q116R and N127D) in the first extracellular loop to confer resistance to ouabain, and the AHC mutation T378N. HeLa cells transfected with this construct did not survive to 1 μM ouabain treatment. A simulated heterozygous state, as obtained by transfecting equal amount of wild-type and mutant cDNAs, showed intermediate behaviour (FIG. 7).

In addition, as revealed by time course experiments, the mutant isoform show rapid mortality typical of cells lacking Na+/K+ ATPase pump activity (FIG. 8).

### REFERENCES

1. Stewart, W. F., Lipton, R. B., Celentano, D. D. & Reed, M. L. Prevalence of migraine headache in the United States. Relation to age, income, race, and other sociodemographic factors. Jama 267, 64-9. (1992).
2. Lipton, R. B., Stewart, W. F., Diamond, S., Diamond, M. L. & Reed, M. Prevalence and burden of migraine in the United States: data from the American Migraine Study II. Headache 41, 646-57. (2001).
3. Rasmussen, B. K. & Olesen, J. Symptomatic and nonsymptomatic headaches in a general population. Neurology 42, 1225-31. (1992).
4. Henry, P. et al. A nationwide survey of migraine in France: prevalence and clinical features in adults. GRIM. Cephalalgia 12, 229-37; discussion 186. (1992).
5. IHS. Classification and diagnostic criteria for headache disorders, cranial neuralgias and facial pain. Headache Classification Committee of the International Headache Society. Cephalalgia 8, 1-96. (1988).
6. Russell, M. B. & Olesen, J. The genetics of migraine without aura and migraine with aura. Cephalalgia 13, 245-8. (1993).
7. Russell, M. B. & Olesen, J. Increased familial risk and evidence of genetic factor in migraine. BMJ 311, 541-4. (1995).
8. Ulrich, V., Gervil, M., Kyvik, K. O., Olesen, J. & Russell, M. B. Evidence of a genetic factor in migraine with aura: a population-based Danish twin study. Ann Neurol 45, 242-6. (1999).
9. Russell, M. B., Ulrich, V., Gervil, M. & Olesen, J. Migraine without aura and migraine with aura are distinct disorders. A population-based twin survey. Headache 42, 332-6. (2002).
10. Blau, J. N. & Whitty, C. Familial hemiplegic migraine. Lancet 2,1115-6 (1955).
11. Ophoff, R. A. et al. Familial hemiplegic migraine and episodic ataxia type-2 are caused by mutations in the Ca2+ channel gene CACNL1A4. Cell 87, 543-52. (1996).
12. Ducros, A. et al. Mapping of a second locus for familial hemiplegic migraine to 1q21-q23 and evidence of further heterogeneity. Ann Neurol 42, 885-90. (1997).
13. Liu, Y., Peterson, D. A., Kimura, H. & Schubert, D. Mechanism of cellular 3-(4,5-dimethylthiazol-2-yl)-2,5-diphenyltetrazolium bromide (MTT) reduction. J Neurochem 69, 581-93. (1997).
14. Hu, Y. K. & Kaplan, J. H. Site-directed chemical labeling of extracellular loops in a membrane protein. The topology of the Na,K-ATPase alpha-subunit. J Biol Chem 275,19185-91. (2000).
15. Price, E. M., Rice, D. A. & Lingrel, J. B. Structure-function studies of Na,K-ATPase. Site-directed mutagenesis of the border residues from the H1-H2 extracellular domain of the alpha subunit. J Biol Chem 265, 6638-41. (1990).
16. Underhill P A, Jin L, Lin A A et al. Detection of numerous Y chromosome biallelic polymorphisms by denaturing high-performance liquid chromatography. Genome Res. 7,996-1005 (1997).
17. Orita M, Suzuki Y, Sekiya T, Hayashi K. Rapid and sensitive detection of point mutations and DNA polymorphisms using the polymerase chain reaction. Genomics. 5, 874-879 (1989).
18. Myers R M, Fischer S G, Lerman L S, Maniatis T. Nearly all single base substitutions in DNA fragments joined to a GC-clamp can be detected by denaturing gradient gel electrophoresis. Nucleic Acids Res. 13, 3131-3145 (1985).

1. Nucleic acid comprising at least one segment of the gene encoding a functional portion or the gene-regulating region of the alpha 2 subunit of the Na,K pump (ATPase, ATP1A2) for use in the diagnosis of pathologies associated with migraine or with alternating hemiplegia of the childhood.

2. Nucleic acid comprising at least one segment of the gene encoding a functional portion or the gene-regulating region of the alpha 2 subunit of the Na,K pump (ATPase, ATP1A2) for use in genetic therapy for pathologies associated with migraine or with alternating hemiplegia of the childhood.

3. Method to detect in an individual at least one mutation in the gene encoding the alpha 2 subunit of the Na,K human pump (ATPase, ATP1A2) located on chromosome1, associated with migraine disorders, which comprises the steps of:
collecting a sample containing a sufficient quantity of the individual's DNA or that is reproducible in culture;
isolating of the DNA from the sample;
exponential amplifying the DNA using as an oligonucleotide pair for the amplification reaction at least two oligonucleotides that are able to amplify at least one segment of the gene encoding the alpha 2 subunit of the Na,K human pump (ATPase, ATP1A2) or a segment of the region regulating it;
detecting in at least one amplified segment any mutations compared with a healthy control.

4. Method according to claim 3 in which the oligonucleotide pairs are:
17 AGTCCCTCTGACCTCCCTGAT CCACTGTGCCATCACGATT
19 CTTCTGCTTCCTGCTCTGACC ACACATGTGCGCTGTGTTTAC.

5. Method according to claim 3 in which the DNA exponential amplification phase is performed using oligonucleotide pairs that are able to amplify the entire encoding portion of the gene encoding the alpha 2 subunit of the Na,K human pump (ATPase, ATP1A2).

6. Method according to claim 5 in which the DNA exponential amplification phase to amplify the entire portion encoding the gene for the alpha 2 subunit of the Na,K human pump (ATPase, ATP1A2) comprises the use of at least one of the following oligonucleotide pairs:

| | | |
|---|---|---|
| 1 | TGTTGCTTTGGCTTTCTCTGT | CTCCCTCACCCTCTAGACTGC |
| 2 + 3 | CCCCTCTCTTCCCTGACTCT | GCCTCTTTTGTTCCTTCCCTA |
| 4 | ATGGTGACTGGCTGGGTTG | CAGGGTTGGAGGACAGTCAC |
| 5 | AGCTGCCCCTTTAGGGTTG | ACCTTACAGCCTAGCCCAGAG |
| 6 | GAGACCAGCAGGAGAAGAAGG | AGACTCAACTGCTTGCTCTGG |
| 7 | TACAAGTGGCTCTGCCAGTCT | AGCCCTTCATCCTGACTATGG |
| 8 | CAGGAAATAGGATGGGACTGC | GTAGTGAGACCCTCCCCTGGT |
| 9 | ATCTCCGGCTTCAGCCTTAAC | TAATCCTATCCACCCCCTCTG |
| 10 + 11 | CTCCTGGTTCCCCCTCAT | TCCCTCTCTCTTCCTCTGTCC |
| 12 | GCGCTACCAAGACAAGTATGG | CTTGGGAATCCCCTTCTGAG |
| 13 | GAAGCCACTCTGCGGATCT | ACTGCAGCTCCTTGAACTCTG |
| 14 | GGAGGGGGATAAACCCTTAAT | GACGTGTTGATTAGGGCACAG |
| 15 | AGGGGTCAGCTGTCTCTGTC | GGTCCCTGCCTGTCA1CTG |
| 16 | AAGGGGTTTCGTCCTCAAGT | TCAGTATCCTGCAAACCATCC |
| 17 | AGTCCCTCTGACCTCCCTGAT | CCACTGTGCCATCACGATT |
| 18 | TCATCTCCTACGTCCCTTCAA | AGCTGGGAAAAGAACCCTGT |
| 19 | CTTCTGCTTCCTGCTCTGACC | ACACATGTGCGCTGTGTTTAC |
| 20 | CCTCCGACACTCTCATCTGTC | CTGTGTGGGTTGGTGAGTGT |
| 21 | CTTCACCTGCCACCTCCTT | CCCCCGTATGACTACTCAGG |
| 22 | CGCTTTGAATGCTCCTTTATG | GAGGGAGGAGCTGGTGGT |
| 23 | GCCTCCTTTTAAGCTCATGCT | GCCTCATTATCTCTCCCCAAA |

7. Method according to claim 3 in which the DNA exponential amplification phase is performed using oligonucleotide pairs that are able to amplify the regulating region of the gene encoding the alpha 2 subunit of the Na,K human pump (ATPase, ATP1A2).

8. Method according to claim 7 in which the DNA exponential amplification phase to amplify the regulating region of the gene encoding the alpha 2 subunit of the Na,K human pump (ATPase, ATP1A2) comprises the use of the following oligonucleotide pairs:
1_Pr TTCCCCTCACTCCATCTCTG GACCCCTGCTCTTTAGGGATA
2_Pr GATTCAGGACCACTCCATCC GGGAACAGTCAGAGGACAGG

9. Method according to claim 3 in which the detection phase of at least one amplified segment with any mutations compared with a healthy control is performed using direct sequencing or an SSCP method (single strand conformation polymorphism) (17) DHPLC or DGGE (denaturing gradient gel electrophoresis) (18).

10. Diagnostic kit for pathologies associated with migraine or with alternating hemiplegia of the childhood to carry out the method according to claims 3, that comprises:
at least one pair of oligonucleotides for the exponential amplification reaction of at least one segment of the gene encoding the alpha 2 subunit of the Na,K human pump (ATPase, ATP1A2), in which the aforesaid segment encodes a functional portion or a gene-regulating portion of the aforesaid subunit; and
a control DNA from a non affected individual.

11. Kit according to claim 10 in which the oligonucleotide pairs for the amplification reaction are able to amplify the entire encoding region of the gene encoding the alpha 2 subunit of the Na,K human pump (ATPase, ATP1A2).

12. Alpha 2 subunit protein of the Na,K human pump (ATPase, ATP1A2) or a functional portion thereof for use in the diagnosis of pathologies associated with migraine or with alternating hemiplegia of the childhood or for use in the treatment of pathologies associated with migraine.

13. (canceled)

14. Method for the identification of an agonist or antagonist agent of the Na,K human pump (ATPase, ATP1A2) or a functional portion or a gene-regulating portion of the subunit, that comprises:
(i) transfection of a cell line with a gene for a mutant isoform of the Na,K human pump (ATPase, ATP1A2) resistant to ouabain;
(ii) appropriate exposure of the transfected cells to the agent;
(iii) measurement of the Na,K pump activity in relation to ion transport with labeled ions.

15. Method for the identification of an agonist or antagonist agent of the Na,K pump (ATPase, ATP1A2) or a functional portion, that comprises the phases:
(i) use of the agent to treat a transgenic animal that expresses a mutant isoform of the Na,K pump (ATPase., ATP1A2) or that is partially or completely deleted in the gene encoding the Na,K pump (ATPase, ATP1A2) or
(ii) use of the agent to treat eukaryotic or prokaryotic cell lines that express mutant or normal forms of the Na,K pump (ATPase, ATP1A2) by transient or stable transfection or in physiological conditions.


SEQUENCE LISTING (46 sequences)

SEQ ID NO: 1 - DNA, 21 bp, Artificial Sequence
  ATP1A2 gene bp177 for
tgttgctttggctttctctgt

SEQ ID NO: 2 - DNA, 21 bp, Artificial Sequence
  ATP1A2 gene bp 177 rev
ctccctcaccctctagactgc

SEQ ID NO: 3 - DNA, 20 bp, Artificial Sequence
  ATP1A2 gene bp 423 for
cccctctcttccctgactct

SEQ ID NO: 4 - DNA, 21 bp, Artificial Sequence
  ATP1A2 gene bp423 rev
gcctcttttgttccttcccta

SEQ ID NO: 5 - DNA, 19 bp, Artificial Sequence
  ATP1A2 gene bp 316 for
atggtgactggctgggttg

SEQ ID NO: 6 - DNA, 20 bp, Artificial Sequence
  ATP1A2 gene bp316 rev
cagggttggaggacagtcac

SEQ ID NO: 7 - DNA, 19 bp, Artificial Sequence
  ATP1A2 gene bp213 for
agctgcccctttagggttg

SEQ ID NO: 8 - DNA, 21 bp, Artificial Sequence
  ATP1A2 gene bp213 rev
accttacagcctagcccagag

SEQ ID NO: 9 - DNA, 21 bp, Artificial Sequence
  ATP1A2 gene bp238 for
gagaccagcaggagaagaagg

SEQ ID NO: 10 - DNA, 21 bp, Artificial Sequence
  ATP1A2 gene bp238 rev
agactcaactgcttgctctgg

SEQ ID NO: 11 - DNA, 21 bp, Artificial Sequence
  ATP1A2 gene bp324 for
tacaagtggctctgccagtct

SEQ ID NO: 12 - DNA, 21 bp, Artificial Sequence
  ATP1A2 gene bp234 rev
agcccttcatcctgactatgg

SEQ ID NO: 13 - DNA, 21 bp, Artificial Sequence
  ATP1A2 gene bp385 for
caggaaataggatgggactgc

SEQ ID NO: 14 - DNA, 21 bp, Artificial Sequence
  ATP1A2 gene bp385 rev
gtagtgagaccctcccctggt

SEQ ID NO: 15 - DNA, 21 bp, Artificial Sequence
  ATP1A2 gene bp283 for
atctccggcttcagccttaac

SEQ ID NO: 16 - DNA, 21 bp, Artificial Sequence
  ATP1A2 gene bp283 rev
taatcctatccaccccctctg

SEQ ID NO: 17 - DNA, 18 bp, Artificial Sequence
  ATP1A2 gene bp487 for
ctcctggttccccctcat

SEQ ID NO: 18 - DNA, 21 bp, Artificial Sequence
  ATP1A2 gene bp487 rev
tccctctctcttcctctgtcc

SEQ ID NO: 19 - DNA, 21 bp, Artificial Sequence
  ATP1A2 gene bp284 for
gcgctaccaagacaagtatgg

SEQ ID NO: 20 - DNA, 20 bp, Artificial Sequence
  ATP1A2 gene bp284 rev
cttgggaatccccttctgag

SEQ ID NO: 21 - DNA, 19 bp, Artificial Sequence
  ATP1A2 gene bp286 for
gaagccactctgcggatct

SEQ ID NO: 22 - DNA, 21 bp, Artificial Sequence
  ATP1A2 gene bp286 rev
actgcagctccttgaactctg

SEQ ID NO: 23 - DNA, 21 bp, Artificial Sequence
  ATP1A2 gene bp236 for
ggagggggataaacccttaat

SEQ ID NO: 24 - DNA, 21 bp, Artificial Sequence
  ATP1A2 gene bp236 rev
gacgtgttgattagggcacag

SEQ ID NO: 25 - DNA, 20 bp, Artificial Sequence
  ATP1A2 gene bp284 for
aggggtcagctgtctctgtc

SEQ ID NO: 26 - DNA, 19 bp, Artificial Sequence
  ATP1A2 gene bp284 rev
ggtccctgcctgtcatctg

SEQ ID NO: 27 - DNA, 20 bp, Artificial Sequence
  ATP1A2 gene bp284 for
aaggggtttcgtcctcaagt

SEQ ID NO: 28 - DNA, 21 bp, Artificial Sequence
  ATP1A2 gene bp284 rev
tcagtatcctgcaaaccatcc

SEQ ID NO: 29 - DNA, 21 bp, Artificial Sequence
  ATP1A2 gene bp252 for
agtccctctgacctccctgat

SEQ ID NO: 30 - DNA, 19 bp, Artificial Sequence
  ATP1A2 gene bp252 rev
ccactgtgccatcacgatt

SEQ ID NO: 31 - DNA, 21 bp, Artificial Sequence
  ATP1A2 gene bp234 for
tcatctcctacgtcccttcaa

SEQ ID NO: 32 - DNA, 20 bp, Artificial Sequence
  ATP1A2 gene bp234 rev
agctgggaaaagaaccctgt

SEQ ID NO: 33 - DNA, 21 bp, Artificial Sequence
  ATP1A2 gene bp232 for
cttctgcttcctgctctgacc

SEQ ID NO: 34 - DNA, 21 bp, Artificial Sequence
  ATP1A2 gene bp232 rev
acacatgtgcgctgtgtttac

SEQ ID NO: 35 - DNA, 21 bp, Artificial Sequence
  ATP1A2 gene bp236 for
cctccgacactctcatctgtc

SEQ ID NO: 36 - DNA, 20 bp, Artificial Sequence
  ATP1A2 gene bp236 rev
ctgtgtgggttggtgagtgt

SEQ ID NO: 37 - DNA, 19 bp, Artificial Sequence
  ATP1A2 gene bp176 for
cttcacctgccacctcctt

SEQ ID NO: 38 - DNA, 20 bp, Artificial Sequence
  ATP1A2 gene bp176 rev
cccccgtatgactactcagg

SEQ ID NO: 39 - DNA, 21 bp, Artificial Sequence
  ATP1A2 gene bp223 for
cgctttgaatgctcctttatg

SEQ ID NO: 40 - DNA, 18 bp, Artificial Sequence
  ATP1A2 gene bp223 rev
gagggaggagctggtggt

SEQ ID NO: 41 - DNA, 21 bp, Artificial Sequence
  ATP1A2 gene bp206 for
gcctccttttaagctcatgct

SEQ ID NO: 42 - DNA, 21 bp, Artificial Sequence
  ATP1A2 gene bp206 rev
gcctcattatctctccccaaa

SEQ ID NO: 43 - DNA, 20 bp, Artificial Sequence
  regulating region ATP1A2 gene, 1 for
ttcccctcactccatctctg

SEQ ID NO: 44 - DNA, 21 bp, Artificial Sequence
  regulating region ATP1A2 gene, 1 rev
gacccctgctctttagggata

SEQ ID NO: 45 - DNA, 20 bp, Artificial Sequence
  regulating region ATP1A2 gene, 2 for
gattcaggaccactccatcc

SEQ ID NO: 46 - DNA, 20 bp, Artificial Sequence
  regulating region ATP1A2 gene, 2 rev
gggaacagtcagaggacagg